(19) United States
(12) Patent Application Publication
(10) Pub. No.: US 2014/0202336 A1
(43) Pub. Date: Jul. 24, 2014

(54) Systems And Method For Air Destratification And Circulation

(71) Applicant: Zoo Fans, Inc., Boulder, CO (US)

(72) Inventors: Jeffrey G. Benton, Boulder, CO (US); Cory R. Cochran, Olathe, KS (US)

(73) Assignee: Zoo Fans, Inc., Boulder, CO (US)

(21) Appl. No.: 14/161,603

(22) Filed: Jan. 22, 2014

Related U.S. Application Data
(60) Provisional application No. 61/755,788, filed on Jan. 23, 2013.

(51) Int. Cl. F24F 11/00; A61L 9/20; B01D 46/00

(52) U.S. Cl. CPC F24F 11/0001; B01D 46/0028; A61L 9/20
USPC 96/224; 454/251; 454/239; 250/430

(57) ABSTRACT

A system for air destratification, circulation, and/or purification including an intake positioned proximate a ceiling of a space formed by a floor, a plurality of walls and the ceiling; an exhaust, positioned proximate the ceiling and separate from the intake; a fan structure comprising a motor mechanically coupled with fan blades; a first duct for coupling the intake to the fan structure; a second duct for coupling the exhaust to the fan structure; and at least one attachment for supporting the fan structure such that no additional stress is applied to the ceiling from the fan structure; wherein the fan structure moves air from the intake to the exhaust via the first and second ducts. The system may be capable of fitting within a single ceiling tile of a T-bar ceiling.

### RELATED APPLICATIONS

This application claims priority to U.S. Provisional Application Ser. No. 61/755,788, filed Jan. 23, 2013, and which is incorporated by reference in its entirety herewith.

### BACKGROUND

Fans have frequently been used to circulate air within a space. Most variable speed controllable fans use either a TRIAC controller, a capacitor-based controller, or a Variable Frequency Drive (VFD) controller to control the fan speed. However, these controller types are not directly suitable for use with an externally wound permanent split-capacitor (PSC) motor. For example, a TRIAC controller is not typically suitable for use with an externally wound PSC motor because the TRIAC introduces voltage spikes that can damage the externally wound PSC motor. These voltage spikes from the TRIAC must be filtered out (for example using a diode) to prevent damage to the windings of an externally wound PSC motor that would reduce the operational life of the motor. Further, at lower speeds, the TRIAC may introduce noise to the system in the form of motor hum. Capacitor-based controllers typically operate fans at stepped speeds, and do not support continuously variable speed control. In addition, capacitor-based controllers require specialized electronic components to prevent externally wound PSC motors from overheating at lower speeds.

FIG. 1 shows a prior art circuit diagram that uses a TRIAC 108 to control a PSC motor 102 having a primary winding 104 and an auxiliary winding 106. The TRIAC changes the trigger or phase angle for the AC voltage, reducing speed by reducing the RMS voltage applied to the PSC motor 102. However, the speed of the motor does not change linearly with the phase angle, and this nonlinearity makes accurate speed adjustments difficult.

Variable Frequency Drive (VFD) controllers, such as the one used in U.S. Patent Publication No. 2010/0291858 to Toy (hereinafter “Toy), incorporated herein by reference, vary the speed of certain types of motors without damaging them. However, VFD's are not compatible with externally wound PSC motors without requiring additional, expensive components to operate the externally wound PSC motor without damage caused by voltage peaks, bearing arcing, and a variety of other issues. Therefore, the life of an externally wound PSC motor is dramatically reduced by direct use of VFD type controllers without expensive components such as all-pole sinusoidal filters.

Certain spaces and buildings are not suitable for traditional, open-bladed ceiling fans and other means of circulation. For example, offices, retail facilities, conference rooms, auditoriums and other spaces with drop ceilings—or T-bar ceilings—are not typically suitable for using ceiling fans that are suspended below the ceiling and use hub-mounted rotating paddles or foils to circulate air. Such ceiling fans cannot be located where the ceiling height results in the moving blades of the fan being a danger to people or other objects within the space through direct or indirect contact, interference with fire suppression equipment, or by creating a strobe effect with lighting.

HVAC systems are sometimes noisy and often do not promote adequate air destratification and circulation within a structure. It is much less efficient to continuously run the central air handler(s) of an HVAC system than it is to run a number of smaller, more efficient fans. Installing an HVAC system as a retrofit (i.e., including new ductwork, intakes and diffusers) has a large project impact (i.e., engineering, logistics, and associated costs). Therefore, retrofitting an HVAC system is not cost effective for addressing localized problems with air destratification and circulation.

### BRIEF DESCRIPTION OF THE FIGURES

FIG. 1 shows a prior art circuit diagram that uses a TRIAC to control a PSC motor having a primary winding and an auxiliary winding.

FIG. 2 is a top view showing one exemplary system for air destratification and circulation, in one embodiment.

FIG. 3 shows a below ceiling view of the system of FIG. 2.

FIG. 4 shows an intake end view of the system of FIG. 2.

FIG. 5 shows an exhaust end view of the system of FIG. 2.

FIG. 6 shows a side view of the system of FIG. 2.

FIG. 7 shows a view of the other side of the system of FIG. 2.

FIG. 8 shows an above ceiling perspective view of the system of FIG. 2.

FIGS. 9 and 10 show one exemplary air destratification control system, in an embodiment.

FIG. 11 shows one exemplary air destratification control system for controlling three destratification fans, in an embodiment.

FIG. 12 shows one exemplary in-ceiling modular destratification system for destratifying air within a space, in an embodiment.

FIG. 13 shows one exemplary method for destratifying air within a space, in one embodiment.

FIG. 14 shows an exemplary combination supply/return directional diffuser, from above.

FIG. 15 depicts a view of the combination supply/return diffuser of FIG. 14 from beneath a ceiling, when the diffuser is installed in the ceiling.

FIG. 16 is a side view of the combination supply/return directional diffuser of FIG. 14.

FIG. 17 depicts an exemplary destratification system installed in a single ceiling tile, from a top perspective view.

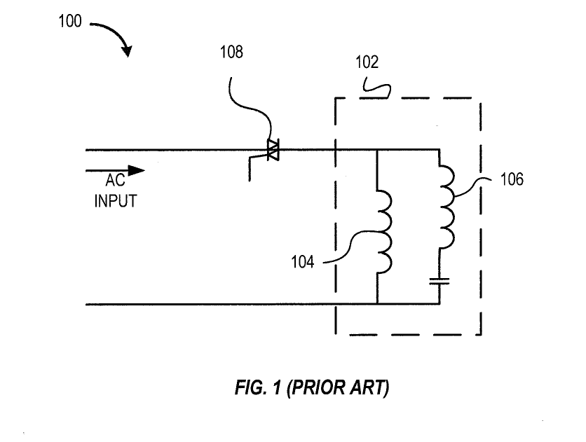

### DETAILED DESCRIPTION OF THE EMBODIMENTS

The embodiments discussed below depict systems and methods for indoor destratification and air circulation. Certain embodiments depict a modular, in-ceiling, independent air mixing and distribution system designed to significantly reduce or eliminate localized temperatures gradients.

Modular, In-Ceiling, Independent Air Mixing and Distribution System;

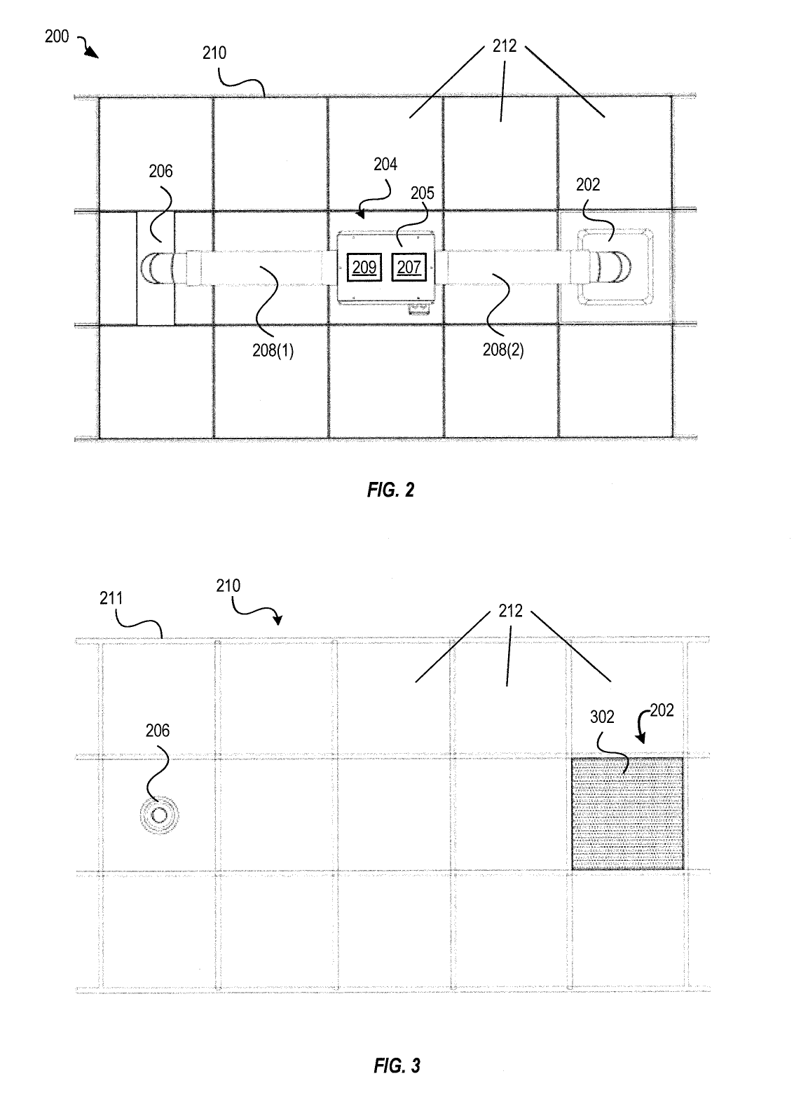

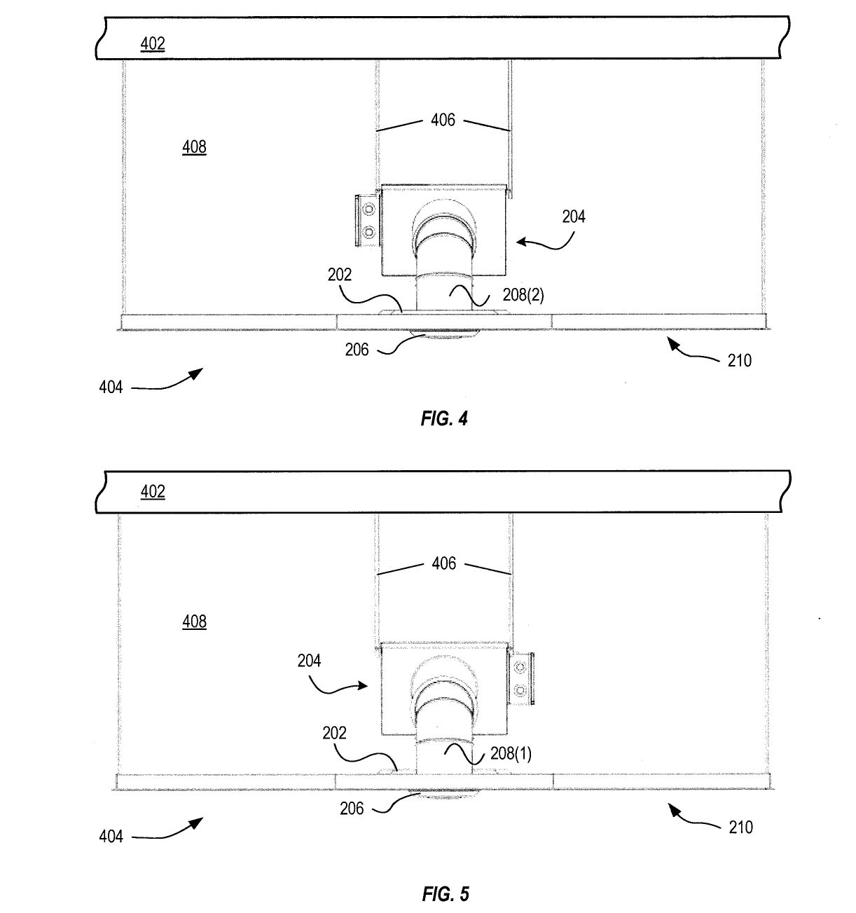

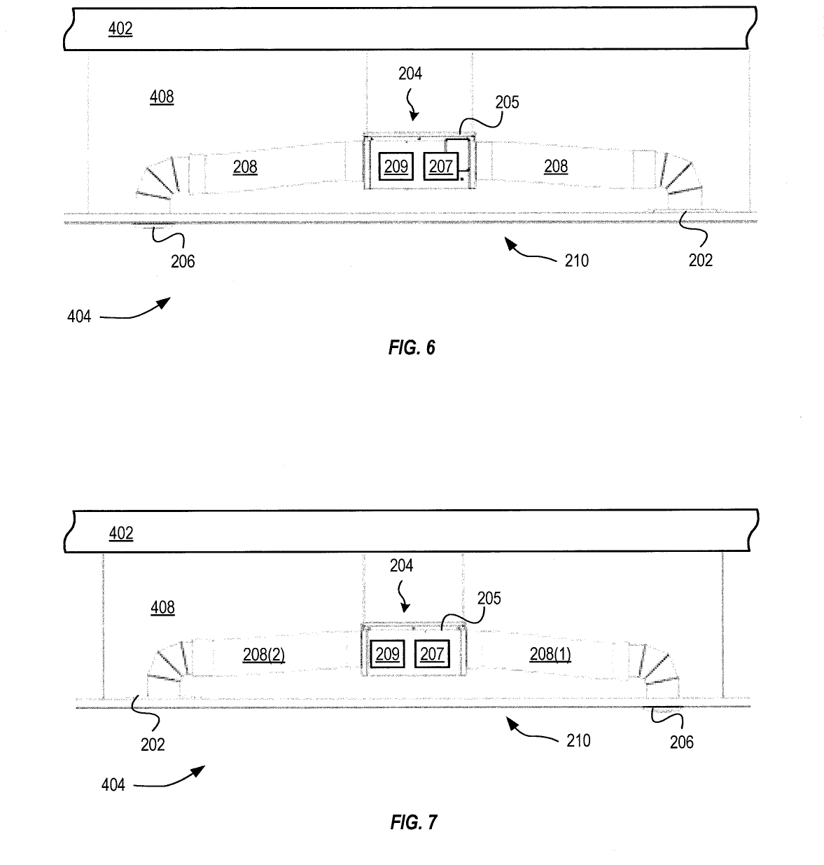

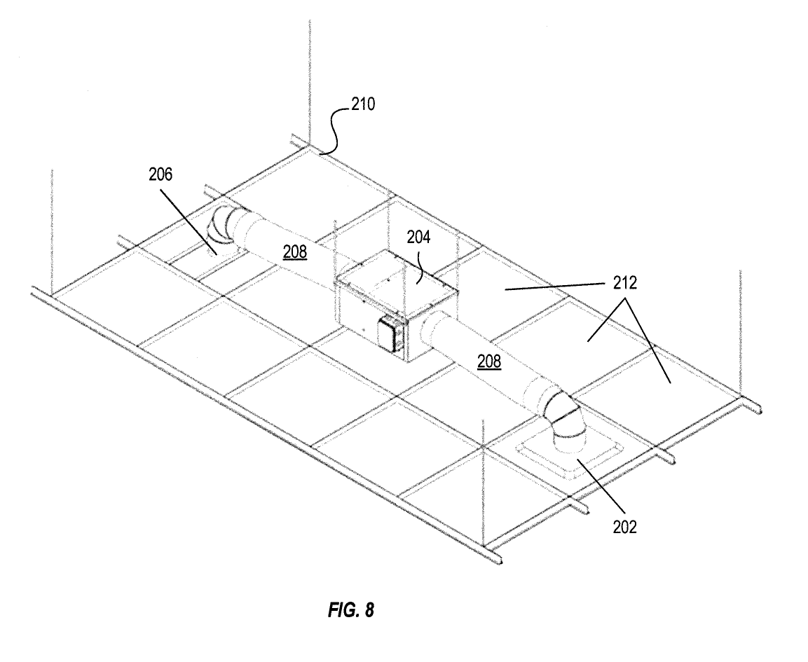

FIG. 2 is a top view showing one exemplary system 200 for air destratification and circulation, in one embodiment. FIG. 3 shows a below ceiling view of system 200 of FIG. 2. FIG. 4 shows an intake end view of system 200 of FIG. 2. FIG. 5 shows an exhaust end view of system 200 of FIG. 2. FIG. 6 shows a side view of system 200 of FIG. 2. FIG. 7 shows a view of the other side, as compared to the view of FIG. 6, of system 200 of FIG. 2. FIG. 8 shows an above ceiling perspective view of system 200 of FIG. 2. In the examples of FIGS. 2 through 8, ceiling 210 is a drop ceiling formed by a plurality of ceiling tiles 212 positioned within a ceiling frame 211; this creates a ceiling area 408 above ceiling 210 and a space 404 below ceiling 210. FIGS. 2-8 are best viewed together with the following description.

System 200 includes an intake 202, an exhaust 206, and an inline fan structure 204 that is positioned in area 408 above a ceiling 210. Ducting 208(2) couples intake 202 to fan structure 204 and ducting 208(1) couples fan structure 204 to exhaust 206. Optionally, in-line fan structure 204 and/or the associated components (e.g. fan 207 and/or UVGI system 209) is specifically engineered for quiet operation, and ducting 208 comprises acoustical features that reduce the sound created by system 200.

Intake 202 includes an inlet grille 302 that may replace at least part of ceiling 210. For example, inlet grille 302 replaces at least a part of one or more ceiling tiles 212 and that is thereby supported by frame 211. Intake 202 may be an intake selected from the group of intakes consisting of: perforated grille, perforated filter grille, T-Bar return air grille, return diffuser, return grille, return air grille, return air filter grille, bar-style grille, bar-style filter grille, egg crate return grille, egg crate filter grille, and any other intake known in the art. For example, where the intake 202 is a perforated grille, intake 202 may include an intake grille 302 that includes a perforated face that is mounted in a downwardly-disposed configuration and coplanar with adjacent ceiling tiles 212. Optionally, intake 202 incorporates an air filter to filter air during operation of system 200. Intake 202 may be positioned proximate ceiling 210 by being mounted either directly within ceiling 210 or by being wall mounted near the ceiling.

System 200 may include one or more additional intakes 202 that couple with fan structure 204 to collect air from space 404. The term “space” as used herein includes any area within a structure having a floor, ceiling and walls. For example, “space” can be any room, office area, retail area, workshop, warehouse, hangar, auditorium, theater, etc. Where multiple intakes are included, ducting 208 may include one or more valves to control airflow from each intake 202. The one or more valves to control airflow from each intake may be manually controlled, or they may be electronically controlled by system 200.

As illustrated in FIGS. 4 and 5, fan structure 204 is supported from building structure 402 by attachments 406 and is thereby not mechanically dependent upon ceiling 210 for support. Attachments 406 may be one or more of: wires, cables, threaded rods, braces, and bars, or any other support mechanism able to support fan structure 204 from building structure 402. Accordingly, the fan structure 204 is supported independent of ceiling 210 and framing 211 and no weight is applied to ceiling 210 or framing 211 from fan structure 204. Sagging of the ceiling 210 as a result of system 200 is thus avoided. In an alternate embodiment, fan structure 204 is supported from ceiling 210 by attaching directly to framing 211 wherein attachments 406 attach to framing 211 and structure 402 to provide the additional support needed to support the additional stress that is applied to ceiling 210 or framing 211 from fan structure 204 such that any additional weight/stress is offset [or “mitigated”]. Further, attachments 406 are not limited in scope to the locations as illustrated, for example, more or fewer attachments may be implemented or there may be attachments associated with other components such as the exhaust, intake ducting, UVGI and/or other components.

Fan structure 204 has a housing 205 that encloses a fan 207. Optionally, fan structure 204 also includes an ultraviolet germicidal irradiation (UVGI) device 209 for purifying the air flowing through fan structure 204. UVGI device 209 is optionally entirely separate from fan structure 204 within system 200. Optionally, housing 205 incorporates acoustic properties to reduce sound created by fan 207. Fan 207 is for example a motor driving a plurality of blades for moving air through system 200. For the purposes herein, blades are intended to mean any element used to move air through the system such as impellers and/or foils. Accordingly, blades may be used interchangeably herein with the terms impellor and foil without departing from the scope hereof. The blades of fan 207 may be axial, mixed-flow or centrifugal relative to the motor without departing from the scope hereof. The motor may be an externally wound Permanent Split-Capacitor (PSC) motor, an Electrically Commutated (EC) motor, or a Direct Current (DC) motor.

Exhaust 206 is selectively configured to provide air circulation and/or air destratification. Exhaust 206 is an exhaust selected from the group of exhausts consisting of: jet diffuser, ball diffuser, supply diffuser, grille, linear bar diffuser, linear slot diffuser, fixed pattern diffuser, curved blade diffuser, nozzle diffuser, supply register and any other exhaust common in the art. For example, exhaust 206 may be configured with a ball diffuser to disperse air received from fan structure 204, via ducting 208(1), into space 404 below ceiling 210. The ball diffuser allows for air to be directed within space 404. Exhaust 206 may be positioned proximate ceiling 210 by being mounted either directly within ceiling 210 or by being wall mounted. For example, Exhaust 206 may be wall mounted near the ceiling in one embodiment or located anywhere away from the ceiling in another embodiment.

Optionally, system 200 includes at least one additional exhaust 206 and corresponding ducting 208 to distribute air from fan structure 204 into space 404. Where multiple exhausts 206 are included, ducting 208(1) may include one or more splits or valves to control airflow to each of the exhausts. The one or more valves to control airflow to each of the exhausts may be manually controlled, or they may be electronically controlled by system 200, via a building management system (BMS), or a combination of both system 200 and a BMS.

Ducting 208 allows the spatial distance between intake 202 and exhaust 206 to be varied. For example, FIG. 3 illustrates an exhaust and intake separated by three ceiling tiles. However, length of ducting 208 may be varied based upon a desired distance between exhaust 206 and intake 202. Ducting 208 is rigid and/or flexible, insulated, non-insulated, and/or acoustic, or a combination thereof, thereby facilitating installation of system 200. Varying the length of the rigid and/or flexible ducting additionally provides the ability for the distance between intake 202 and exhaust 206 to be lengthened or shortened, thereby providing customization based upon specific installation requirements.

In another embodiment, system 200 may be implemented in an “all-in-one” system which is capable of replacing only a single ceiling tile. For example, the system described herein may utilize a supply/return directional diffuser as disclosed in U.S. Provisional Application Ser. No. 61/928,254, entitled “COMBINATION SUPPLY/RETURN DIRECTIONAL DIFFUSER, AND ASSOCIATED SYSTEM FOR ADJUSTABLY CIRCULATING AIR WITHIN A SPACE”, filed Jan. 6, 2014 by Jeffrey Benton, et al, and which is incorporated by reference herewith.

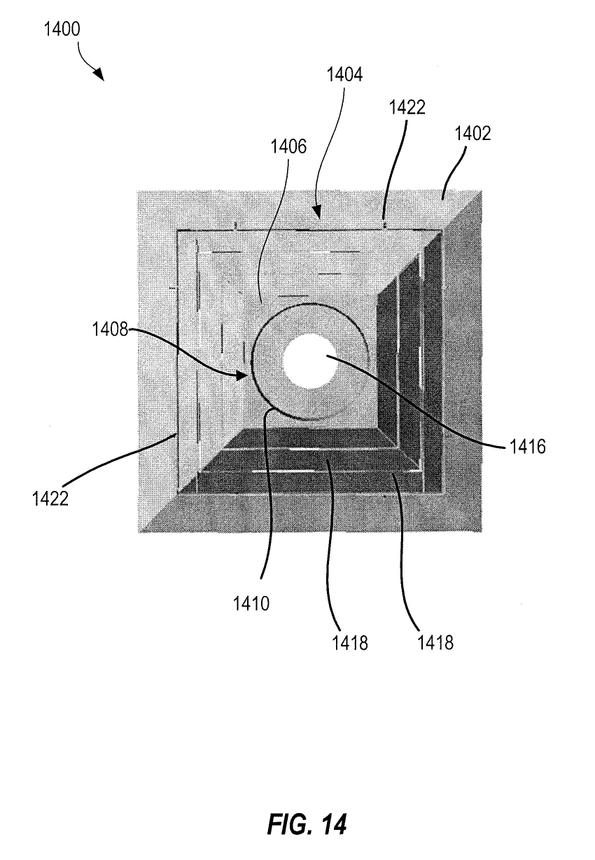

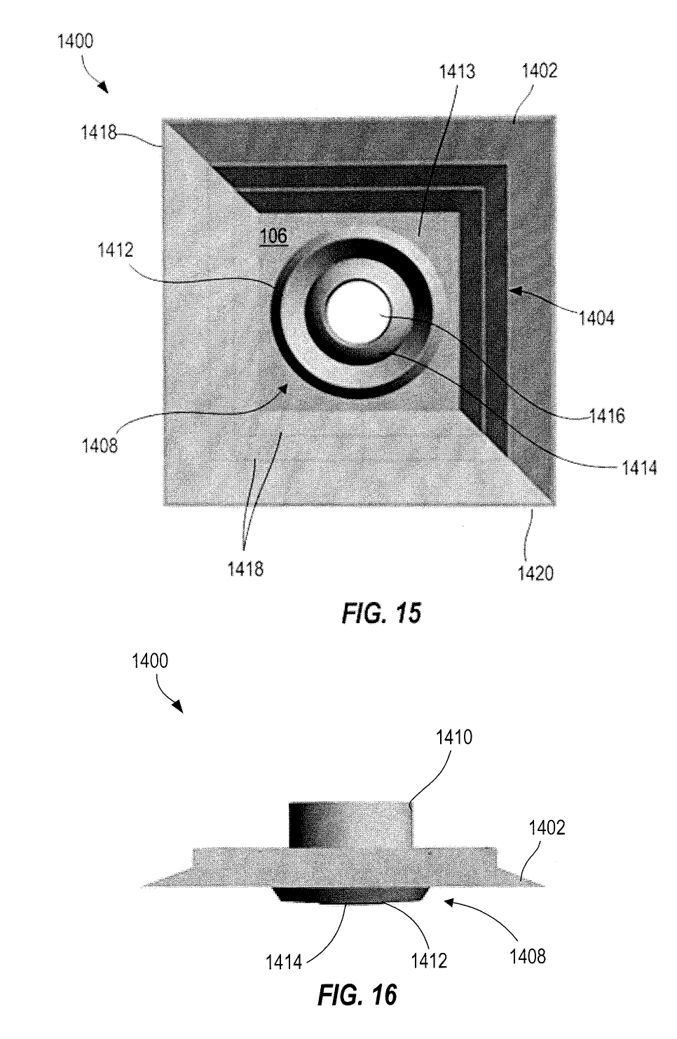

FIG. 14 shows a combination supply/return directional diffuser 1400 from above. FIG. 14 for example shows the diffuser as may be seen from above a ceiling, when the diffuser is installed in a standard 2′×2′ section of a suspended ceiling grid. FIG. 15 shows supply/return directional diffuser 1400 from below. For example, FIG. 15 represents the view of supply/return directional diffuser 1400 from beneath a ceiling, when the diffuser is installed in the ceiling. FIG. 16 is a side view of supply/return directional diffuser 1400. FIGS. 14-16 are best viewed together with the following description:

Supply/return directional diffuser 1400 includes a housing or frame 1402 (e.g., the open housing/frame of the attached Figures), an intake 1404 supported with frame 1402, a central plate 1406 and a central, directionally-adjustable air outlet 1408 disposed central to intake 1404. Thus, supply/return directional diffuser 1400 provides intake 1404 and outlet 1408 within a common housing. Frame 1402, intake 1404, plate 1406 and outlet 1408 may be metal, plastic, other structurally supportive materials or combinations thereof.

Outlet 1408 includes a neck 1410 (see especially FIG. 16) for connecting with a fan, ductwork or HVAC component (i.e., an in-ceiling component, not shown), a lower, outer collar 1412 extending from a bottom side 1413 of central plate 1406, and an exemplary moveable jet-ball 1414 supported within collar 1412. Jet-ball 1414 may be rotated or swiveled within collar 1412 to selectively direct a column of air from the fan through an aperture 1416. Columnar output reduces lateral dispersion, thus increasing the delivery range of air through outlet 1408 and enhancing the vacuum effect that draws air upward and through intake 1404. Supply/return directional diffuser 1400 thereby mixes air more effectively over a longer distance, improving overall air circulation and destratification. It will be appreciated that in addition to use with heating and/or air conditioning systems, supply/return directional diffuser 1400 may also provide adjustable control and improved circulation of air output from air purification, air humidification or other air treatment systems.

Jet-ball 1414 may be angled to direct the column of discharge air, for example to avoid blowing the air directly on people or objects below. Supply/return directional diffuser 1400 may be used with existing HVAC systems, or with closed loop, intake/exhaust systems. As supply/return directional diffuser 1400 is itself adjustable, it may advantageously serve as a retrofit to non-adjustable HVAC or other systems in order to allow for customized direction of air discharged from an otherwise non-adjustable system. For example, when installed with an above-ceiling system, supply/return directional diffuser 1400 may be accessed from below the ceiling and jet ball 1414 may be manually or mechanically adjusted to direct the column of discharge air into a space beneath the ceiling as may be desired at any time post-installation.

Supply/return directional diffuser 1400 may attach with ducting of a centralized HVAC system (i.e., via neck 1410) such that jet ball 1414 increases throw of airflow supplied via the system ducting. Increased airflow and adjustability of direction of flow (by adjusting jet ball 1414) improves overall air circulation and increases user customization of airflow direction. In the case of open plenums above drop ceilings, the intake area around the outlet in the diffuser (i.e., intake 104) may be left open above the ceiling to allow for return air. This provides a heretofore unavailable means to replace a standard 2′×2′ intake with an additional, directional outlet without disrupting existing supply and return balancing.

Intake 1404 is shown with a plurality of directional louvers 1418 disposed about jet ball 1414/outlet 1408. Among other advantages, louvers 1418 enhance the appearance of supply/return directional diffuser 1400 and are angled to reduce or prevent recirculation of discharge air. The angle of louvers 1418 may also discourage dust collection upon supply/return directional diffuser 1400. It will be appreciated that intake 1404 may include more or fewer louvers without departing from the scope hereof. It will also be appreciated that intake may alternately include an egg-crate core, mesh or other acceptable intake material.

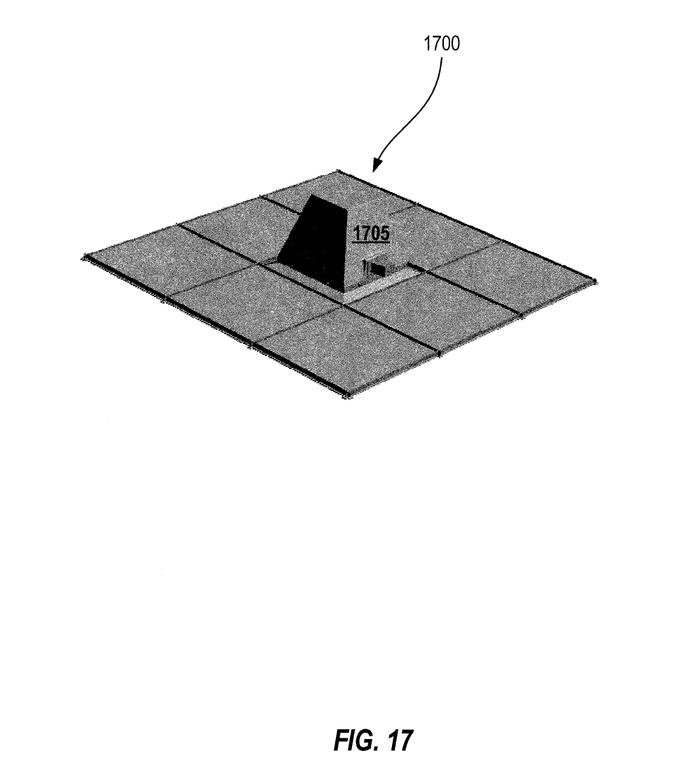

FIG. 17 depicts an exemplary destratification system 1700 installed in a single ceiling tile 1702, from a top perspective view. System 1700 includes combination supply/return diffuser 1400, described above. Moreover, system 1700 includes a housing 1705 for housing a fan, for example controlled by one of an externally wound PSC motor, an EC motor, or a DC motor as discussed above with reference to FIG. 2. System 1700 may include additional elements of system 200 discussed above without departing from the scope hereof. For example, system 1700 may include, all within housing 1705, a UVGI system (similar to system 209), intake filters or other elements. Additionally, housing 1705 may be made of materials that are insulated, non-insulated, acoustic, or a combination thereof. Furthermore, system 1700 may include attachments, similar to attachments 406 to aide in the installation of 1700.

System 200 may be used to destratify, circulate, and/or purify air within multiple spaces by positioning exhaust 206 and intake 202 proximate each of the spaces (i.e., within the ceiling, within the walls or within the floor). System 200 operates independently from traditional HVAC systems and may be custom designed for each particular application. The use of independent support attachments 406 reduces stress on already constructed ceilings (e.g., ceiling 210), thereby allowing easy installation. Furthermore, by configuring acoustic properties of ducting 208 and housing 205, the amount of noise produced during operation of system 200 may be significantly reduced to allow use of system 200 within sound-sensitive spaces.

Externally Wound Permanent Split-Capacitor (PSC) Motor Controller:

The embodiments below describe an air destratification system with a controller for controlling speed of a fan having an externally wound PSC motor. Although the below description mainly discusses a destratification system, it should be acknowledged to a person of ordinary skill in the art that the same principles can be applied for circulation and/or purification systems as well.

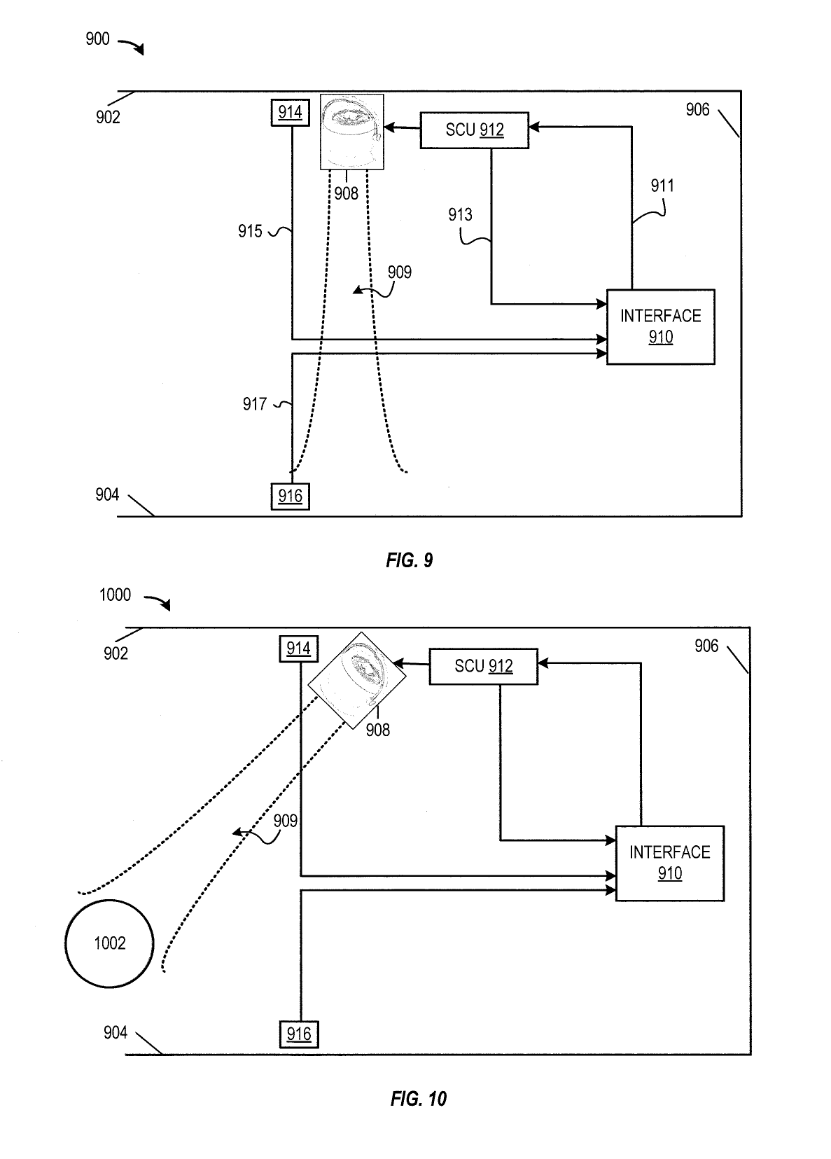

FIGS. 9 and 10 show an exemplary air destratification control system 900 for controlling a destratification fan 908. Destratification control system 900 includes an interface 910, a destratification fan speed control unit 912, a first temperature sensor 914, and a second temperature sensor 916.

Destratification fan 908 includes an externally wound PSC motor driving axial, mixed-flow, or centrifugal blades. In operation, destratification fan 908 generates an air column 909, which may be directed towards a target area 1002, and moves air from a first level (e.g., proximate the ceiling of a space) to a second level (e.g., proximate the floor of the space). The term “air column” as used herein refers to the general shape of the air generated. “Air column” is a column of moving air (i.e., an air jet) and is not limited to a precise column but may gradually expand outward as the distance from the fan increases. “Air column” is not limited to a vertical column of moving air, but additionally incorporates a horizontal column of moving air, or for example a column of moving air at any angle with respect to the surrounding space. Targeted area 1002 may be the floor beneath fan 908, as depicted in FIG. 9 or another location within the space (e.g., an area away from a person's desk), as illustrated in FIG. 10.

First temperature sensor 914 is positioned to sense air temperature proximate a ceiling of a space (e.g., proximate destratification fan 908). Second temperature sensor 916 is positioned to sense air temperature proximate to the floor (e.g., beneath destratification fan 908). Alternatively, second temperature sensor 916 may be positioned to sense air temperature proximate to a point of interest 1002. First and second temperature sensors 914, 916 may be positioned elsewhere within the space to sense any temperature differential without departing from the scope hereof. For example, first and second sensors 914, 916 may be placed on the walls to sense horizontal differential within the space.

Temperature sensors 914 and 916 generate temperature signals 915 and 917, respectively, which are received by interface 910. Interface 910 analyzes signals 915, 917 and generates a speed control signal 911 for controlling the speed (e.g., rotational speed) of destratification fan 908, thereby controlling airflow within air column 909. Speed control signal 911 is for example an electrical signal having a voltage range of 0-10 VDC to indicate a desired speed for fan 908. For example, where temperature signal 917 indicates a temperature within the programmed temperature variation from signal 915, interface 910 generates speed control signal 911 of 0 VDC, indicating that fan 908 should be turned off. In another example, where temperature signal 915 indicated a temperature that is significantly greater than the programmed temperature difference indicated by temperature signal 917, interface 910 generates speed control signal 911 of 10 VDC, to indicate a maximum speed for fan 908. Although the above discussion discusses the first sensor having a temperature greater than the second sensor, it will be appreciated that the differential may be based on the absolute difference between the two sensors 914 and 916 without departing from the scope hereof. In order to reach and maintain a desired differential between the two sensors, interface 910 generates and sends speed control signal(s) 911 to increase and decrease the speed of fan 908. For example, once a desired differential between the temperature sensors is reached, interface 910 generates and sends speed control signal 911 to reduce the speed of fan 908.

Interface 910 includes one or more algorithms, implemented in one or both of hardware and software, for determining a proportional speed for fan 908 and generates speed control signal 911 with a voltage accordingly. In an alternative embodiment, speed control signal 911 is current based, wherein the current is controlled to indicate a desired speed of fan 908. In another embodiment, speed signal 911 is digital, wherein digital information is generated and transmitted using speed signal 911. Other protocols for sending speed information from interface 910 may be used without departing from the scope hereof.

Interface 910 generates speed control signal 911 based upon one or more of: a difference between temperature signals 915 and 917, a vertical distance between temperature sensors 914 and 916, a height of the ceiling in the space, user input, and input from a building management system input. The height of the ceiling may be used to determine an optimal speed for fan 908 based upon predetermined characteristics of air column 909. For example, ceiling height may define a minimum speed for fan 908 required for air column 909 to reach target area 1002. In another example of operation, a user manually sets a desired speed for fan 908, using either direct input to interface 910 or input from a wireless device such as a remote control or an app located on an electronic device.

In another embodiment, interface 910 includes an interface that couples with the building management system to receive information for controlling fan 908. For example, where a building management system incorporates temperature sensors, interface 910 may receive information from the building management system temperature sensors to control airflow from one space within the building to another space within the building. In yet another embodiment, the BMS is the source of the speed control signal 911, and the speed control signal 911 is sent directly from the BMS to the speed control unit 912.

Speed control unit (SCU) 912 receives speed control signal 911 and controls the speed of the destratification fan 908 accordingly. Where destratification fan 908 incorporates an externally wound PSC motor, SCU 912 incorporates a transformer based speed controller that controls power provided to the externally wound PSC motor based upon speed signal 911. Optionally, SCU 912 provides a feedback signal 913 that is received by interface 910. Feedback control signal 913 indicates a speed of fan 908, for example.

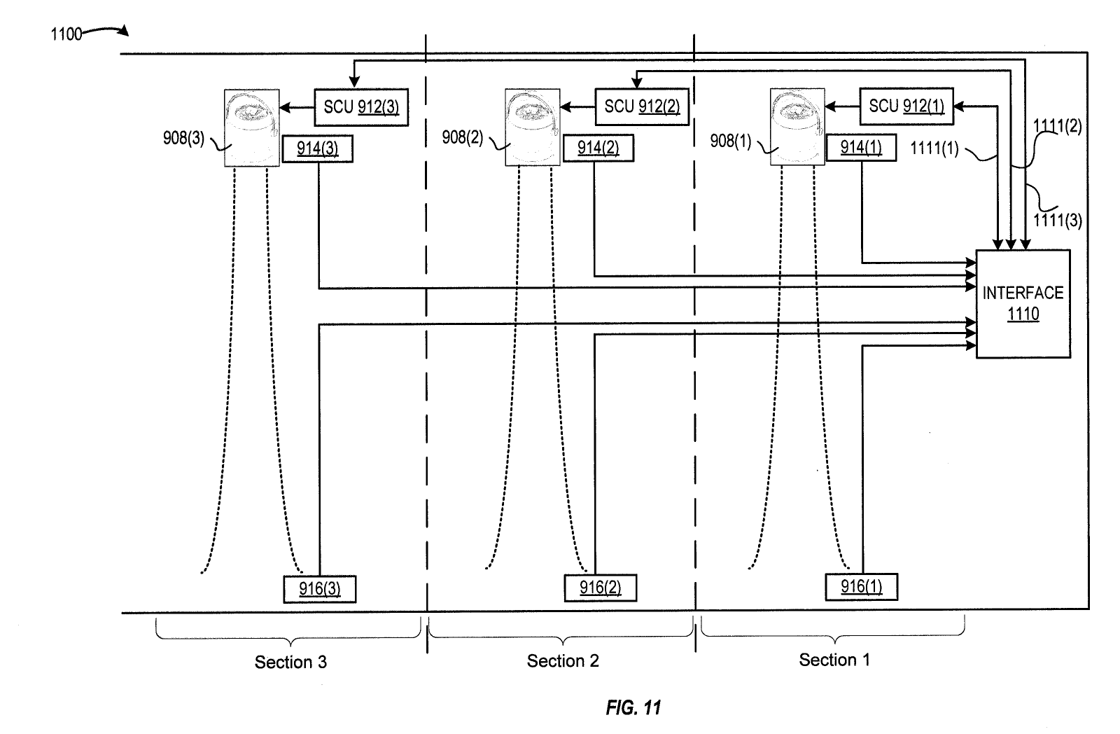

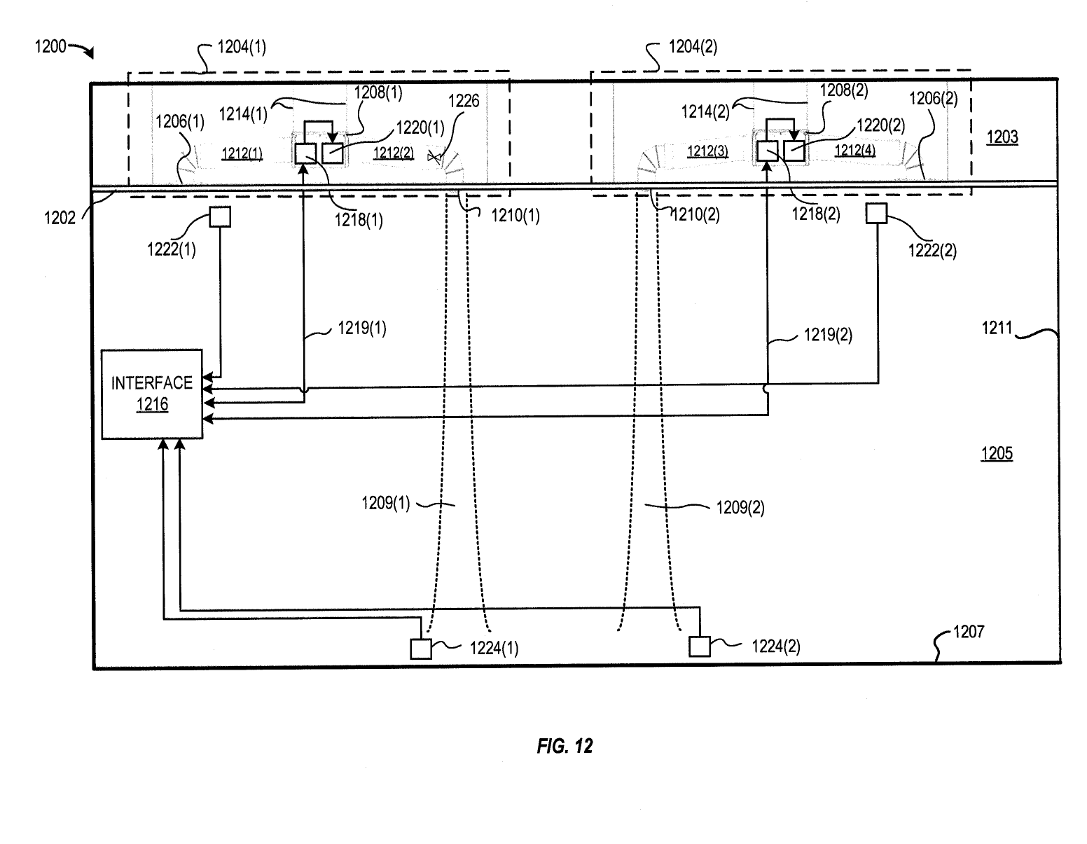

FIG. 11 shows one exemplary air destratification control system 1100 for controlling three destratification fans 908(1)-(3). More or fewer fans may be implemented within system 1100 without departing from the scope hereof. Each destratification fan 908(1)-908(3), has a SCU 912(1)-912(3) that receives a speed control signal 1111(1)-(3), respectively. Optionally, a single SCU 912 may contain multiple outputs to control a plurality of destratification fans 908, or a single SCU 912 may utilize a single output to control a plurality of destratification fans 908. In the example of FIG. 11, a first pair of temperature sensors 914(1) and 916(1) is associated with fan 908(1) and SCU 912(1) within destratification section 1. A second pair of temperature sensor 914(2) and 916(2) is associated with fan 908(2) and SCU 912(2) within destratification section 2. A third pair of temperature sensor 914(3) and 916(3) is associated with fan 908(3) and SCU 912(3) within destratification section 3. Each section of the space may thereby be destratified independently of other sections. Each section may have more fans 908, and temperature sensors 914, 916 without departing from the scope hereof. For example, where multiple fans are located within the same section, their speed is controlled in parallel based upon the sensed temperatures within that section.

System 1100 also includes an interface 1110, similar to interface 910 of FIGS. 9 and 10, which incorporates additional functionality to generate speed control signals 1111(1)-(3) to simultaneously control the speed of fans 908(1)-(3), respectively. Table 1 shows, for each section of the space, exemplary sensed first (higher) and second (lower) temperature values and a value of the associated speed control signal.

TABLE 1
Speed Control Example

| | First Sensor | Second Sensor | |
|---|---|---|---|
| | Temp Reading | Temp Reading | Speed Control |
| Section | (° F.) | (° F.) | Signal Output |
| 1 | 73 | 71 | OFF/LOW |
| 2 | 78 | 70 | MEDIUM |
| 3 | 84 | 66 | HIGH |

In the example of Table 1, section 3 of the space is more stratified than section 1, for example due to fewer windows within section 1 than section 3. Interface 1110 determines that section 1 does not need destratification, since first and second temperature reading for that section are similar and therefore sets speed control 1111(1) to “LOW” (or optionally “OFF”). Interface 1110 determines that a medium fan speed is needed for section 2 based upon a temperature differential of eight degrees in that section. Interface 1110 determines that a “HIGH” fan speed is required for section 3 based upon a sensed temperature differential of eighteen degrees. Interface 1110 thereby generates speed control signals 1111(1)-(3) accordingly to control the speeds of fans 908(1)-(3) using SCUs 912(1)-(3), respectively. The example illustrated in Table 1 depicts a stepped variable speed control signal. However, the embodiments herein are not limited thereto. Accordingly, the speed control signal may be continuously variable instead of stepped variable. Although the above discussion illustrates decisions based on the first sensor temperature reading being greater than the second sensor temperature reading, it should be appreciated that decisions may be based upon the absolute difference between the two sensors as well.

In the examples above, system 900 is shown providing vertical destratification. However, system 900 may provide horizontal temperature equalization, or any other type of temperature equalization, without departing from the scope hereof. For example, temperature sensors 914 and 916 may be horizontally separated and not vertically separated, and the fan(s) placed to move air horizontally with a space. Interface 910 generates speed control signal 911 based on one or more of: the difference between the temperature sensors, the distance from one wall to the other wall, and the distance from the destratification fan 908 to a target area, and user input.

System 900 optionally incorporates wireless capabilities. Where wireless capabilities are incorporated, temperature signals 915 and 917 are transmitted wirelessly to interface 910. Additionally, interface 910 transmits speed control signal 911 wirelessly to speed control unit 912. Accordingly, predetermined logic within speed control unit 912 determines the speed of destratification fan 908 based upon the wirelessly transmitted speed control signal. Speed control unit may transmit the feedback signal wirelessly to interface 910. Further, one or more of the signals from temperature sensors 915, 917, speed control signal 911, and feedback signal 913 may be communicated wirelessly without departing from the scope hereof.

The above examples utilize temperature readings from temperature sensors within interface 910 to control the speed of destratification fan 908. However, additional sensor technology may be implemented without departing from the scope hereof. For example, the system may utilize one or more sensors from the group of sensors including: humidity level sensors, mold level sensors, air particulate sensors, carbon dioxide sensors, carbon monoxide sensors, occupancy sensors, and other air characteristic sensors without departing from the scope hereof.

Controller Combined with In-Ceiling Modular Destratification System:

FIG. 12 shows one exemplary in-ceiling modular destratification system 1200 for destratifying air within a space. System 1200 includes two in-ceiling destratification sections 1204(1) and 1204(2) located within area 1203 above a ceiling 1202. Each destratification section 1204 includes an intake 1206, an inline fan structure 1208, an exhaust 1210, ducting 1212 coupling the intake 1206 to the inline fan structure 1208, ducting 1212 coupling the inline fan structure 1208 to the exhaust 1210, and attachments 1214. Each intake 1206(1), 1206(2), is located on the outer portion of space 1205 formed by ceiling 1202, a floor 1207, and a plurality of walls 1211 (only one of which is labeled for clarity of illustration). Each exhaust 1210(1) and 1210(2) is located on the inner portion of the space 1205. In-ceiling destratification section 1204 may represent system 200 of FIGS. 2-8, for example.

Fan structure 1208 includes a fan 1220 and an SCU 1218. Those skilled in the art will appreciate that SCU 1218 is not limited in scope to being integral to the fan structure 1208, and may be placed separately therefrom. Fan 1220 is similar to fan 220 of FIGS. 2-8 and SCU 1218 is similar to SCU 912 of FIGS. 9-11. Destratification sections 1204(1)-(2) are controlled by an interface 1216 that is similar to interface 910 of FIGS. 9 and 10 and to interface 1110 of FIG. 11. Interface 1216 is communicatively coupled with temperature sensors 1222 and 1224 and operates similar to interface 1110 of FIG. 11 to control speed of fans 1220 based upon sensed temperature differentials within the space.

Although system 1200 is shown with one SCU 1218 for each destratification fan 1220, one SCU 1218 may control the speed of more than one fan 1220 without departing from the scope hereof. Further, speed control units 1218 may be located external to destratification sections 1204 without departing from the scope hereof.

As with system 200, each destratification section 1204 may have more or fewer intakes 1206 and exhausts 1210 without departing from the scope hereof. For example, there may be a single intake 1206 that supplies multiple exhausts 1210. In such an embodiment, interface 1216 may also control one or more valves 1226 such that air destratification occurs only at desired exhausts 1210. Optionally, each intake 1206 and exhaust 1210 may have a respective valve 1226 to control airflow to each particular intake and exhaust. Accordingly, interface 1216 may vary a speed control signal 1219 to control speed of fan 1220 based upon cumulative desired airflow through exhausts 1210. Where fan 1220 is configured with an EC motor (or optionally a DC motor), speed control unit 1218 may be omitted, wherein speed control signal 1219 is generated within interface 1216 (or via the BMS, or a combination of both the BMS and interface 1216), to control the EC or DC motor directly.

In the example of FIG. 12, system 1200 circulates air from the outer portions of the space to the inner portion of the space, and provides columns of air 1209 to destratify air within the space. System 1200 may be implemented with alternative configurations without departing from the scope hereof. For example, intake 1206 may be located in the inner portion of the space and the exhaust 1210 located near the outer portion of the space.

System 1200 destratifies the space without intrusion of destratification section 1204, or fans 1220 thereof, within the controlled environment of the space. System 1200 is particularly useful where the space has a drop ceiling for accommodating destratification sections 1204. Furthermore, destratification system 1200 is independent from the existing HVAC system and therefore is particularly useful as a retrofit to existing structures.

The embodiments described above depict using an externally wound PSC motor. However, the systems above may incorporate an Electrically Commutated motor (EC). Where an EC motor is incorporated, the speed control units are not necessary. The speed control signal generated by the interface may drive the EC or DC motor directly.

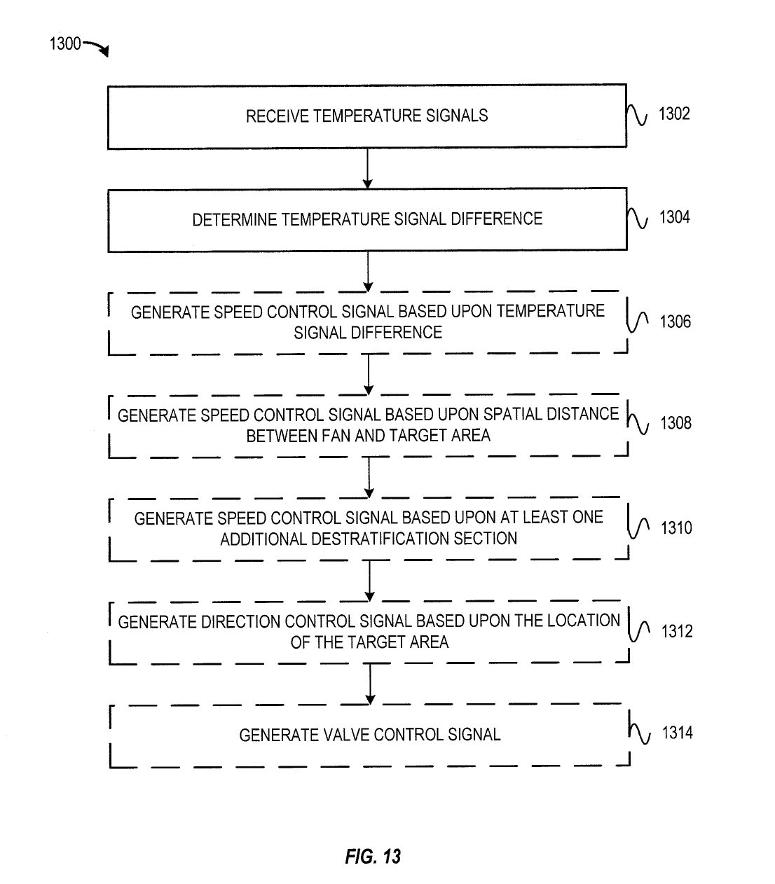

FIG. 13 shows one exemplary method 1300 for destratifying air within a space. For example, method 1300 is implemented using one of the systems depicted in FIGS. 2-12.

In step 1302, an interface receives temperature signals from a plurality of temperature sensors. In one embodiment, interface 1216 receives temperature signals from one or more of temperature sensors 1222(1), 1222(2), 1224(1) and 1224(2). It will be appreciated that there may be more or fewer temperature signals received by the interface in step 1302.

In step 1304, the interface determines a difference between the plurality of temperature signals received. For example, interface 1216 determines the difference between the temperature signals generated from temperature sensors 1224(1) and 1222(1). In one embodiment, the interface determines the difference between temperature sensors that are associated with one another. For example, as illustrated in FIGS. 9-10 a sensor proximate to a destratification fan may be associated with at least one sensor associated with a target area. Alternatively, as illustrated in FIGS. 2-8, and 12, a sensor proximate an intake may be associated with at least one sensor associated with a target area.

In step 1306-1314, the interface generates control signals to control one or more of the speed of the destratification fan, the direction of the destratification fan, and the valves supplying airflow to the exhaust or from the intake. Steps 1306-1314 are optional depending on the application. However a minimum of one of steps 1306-1314 must take place during implementation of method 1300.

For example, in step 1306, interface generates a speed control signal based upon, at least in part, the difference in temperature signals received from the temperature sensors. This speed control signal may be stepped variable, or continuously variable based upon the application. The speed control signal may be sent to a speed control unit, or in the case of an EC motor used directly to control the EC or DC motor.

In step 1308, interface generates a speed control signal based upon, at least in part, a spatial distance between the fan and the target area. The spatial distance may be horizontal or vertical, or any angular distance. For example, the spatial distance is approximately the distance from the floor of a space to the ceiling of a space.

In step 1310, interface generates a speed control signal based upon, at least in part, at least one additional destratification section. Destratification sections may be similar to destratification sections 1-3 as illustrated in FIGS. 11-12. In this step, interface uses information regarding the distance between at least one additional destratification section, and the current destratification section to generate the speed control signal. For example, where two adjacent destratification sections require destratification, the interface may adjust the speed control signal sent to each destratification section accordingly.

In step 1312, interface generates a direction control signal based upon, at least in part, the location of the target area. In this step, the direction of the destratification fan may be altered to point to a particular target area. In one embodiment, destratification fans in one destratification section may be rotated based upon the direction control signal, to point the column of air towards a different destratification section. Accordingly, this may alter the spatial distance between the destratification fan and target area, whereby adjustment of the speed control signal generated in step 1308 may be required. In another embodiment, the direction of an exhaust may be adjusted in a similar manner. In yet another embodiment, the direction of the destratification fan and/or exhaust may additionally be controlled manually.

In step 1314, interface generates a valve control signal. In step 1314, valve control signal controls one or more of an exhaust valve and/or an intake valve, as discussed above with reference to the systems depicted in FIGS. 2-8 and 12.

The above examples utilize temperature readings from temperature sensors within the interface to control the speed of destratification fan. However, additional sensor technology may be implemented without departing from the scope hereof. For example, the system may utilize one or more sensors from the group of sensors including: humidity level sensors, mold level sensors, air particulate sensors, carbon dioxide sensors, carbon monoxide sensors, occupancy sensors, and other air characteristic sensors without departing from the scope hereof.

Combination of Features:

Features described above as well as those claimed below may be combined in various ways without departing from the scope hereof. The following examples illustrate some exemplary combinations:

(A1) A system for air destratification and circulation, the system including: an intake positioned proximate a ceiling of a space formed by a floor, a plurality of walls and the ceiling; an exhaust, positioned proximate the ceiling and separate from the intake; a fan structure including a motor mechanically coupled with fan blades; a first duct for coupling the intake to the fan structure; a second duct for coupling the exhaust to the fan structure; and at least one attachment for supporting the fan structure such that no additional stress is applied to the ceiling from the fan structure; wherein the fan structure moves air from the intake to the exhaust via the first and second ducts.

(B1) In the system described in (A1), wherein the intake comprises an intake chosen from the group of intakes consisting of: perforated grille, perforated filter grille, T-Bar return air grille, return diffuser, return grille, return air grille, return air filter grille, bar-style grille, bar-style filter grille, egg crate return grille, and egg crate filter grille; wherein the intake is configured to replace at least a part of the ceiling.

(C1) In any of the systems described above in (A1) through (B1), wherein the exhaust comprises a diffuser configured to provide a column of air into the space.

(D1) In any of the systems described above in (A1) through (C1), wherein the exhaust is an exhaust chosen from the group of exhausts consisting of: jet diffuser, ball diffuser, supply diffuser, grille, linear bar diffuser, linear slot diffuser, fixed pattern diffuser, curved blade diffuser, nozzle diffuser, and supply register.

(E1) In any of the systems described above in (A1) through (D1), wherein one or more of the fan structure, the first duct and the second duct includes acoustic properties for reducing noise produced by the system.

(F1) In any of the systems described above in (A1) through (E1), further including an ultraviolet germicidal irradiation (UVGI) device to purify air flowing through the system.

(G1) In any of the systems described above in (A1) through (F1), further including an air filter to purify air flowing through the system.

(H1) In any of the systems described above in (A1) through (G1), wherein the motor is an externally wound Permanent Split Capacitor (PSC) motor.

(I1) In any of the systems described above in (A1) through (G1), wherein the motor is one of an Electrically Commutated (EC) motor or a Direct Current (DC) motor.

(J1) In any of the systems described above in (A1) through (I1), wherein the fan blades are one or more of axial blades, mixed-flow blades, or centrifugal blades.

(K1) In any of the systems described above in (A1) through (J1), wherein one or both of the first and second ducts are insulated, non-insulated, rigid or flexible.

(L1) In any of the systems described above in (A1) through (K1), further including at least one additional exhaust coupled with the fan structure.

(M1) In any of the system described above in (L1), further including a valve configured to control airflow through the additional exhaust.

(N1) An air destratification, circulation, and/or purification control system, the system including: a first air characteristic sensor for sensing an air characteristic reading at a first location within a space formed by a ceiling, a floor and a plurality of walls; a second air characteristic sensor for sensing the air characteristic reading at a second location within the space; a user interface unit for generating a speed control signal based upon the air characteristic readings sensed by the first and second air characteristic sensors; and a speed control unit having a transformer based power controller for controlling, based upon the speed control signal, speed of a destratification fan configured with an externally wound permanent split capacitor motor.

(O1) In the system described above in (N1), wherein the first location is proximate the ceiling of the space and the second location is proximate the floor of the space.

(P1) In any of the systems described above in (N1) through (O1), wherein the interface generates the speed control signal based upon a spatial distance between the first and second locations.

(Q1) In any of the systems described above in (N1) through (P1), further including at least one additional destratification fan coupled to the speed control unit.

(R1) In any of the systems described above in (N1) through (Q1), further including a third air characteristic sensor for sensing the air characteristic reading at a third location within the space; a fourth air characteristic sensor for sensing the air characteristic reading at a fourth location within the space; and at least one additional speed control unit having a transformer based power controller for controlling speed of a second destratification fan configured with an externally wound permanent split capacitor motor; wherein the user interface unit generates a second speed control signal based upon the air characteristic readings sensed by the third and fourth air characteristic sensors; and wherein the at least one additional speed control unit controls the speed of the second destratification fan based upon the second speed control signal.

(S1) In any of the systems described above in (N1) through (R1), further including at least one additional destratification fan configured with an electrically commutated (EC) or DC motor; wherein the speed of at least one of the at least one additional destratification fan configured with an EC motor is controlled directly based upon at least one additional speed control signal.

(T1) A system for air destratification and circulation, including: a combination supply/intake diffuser positioned in a ceiling of a space having a supply surrounded by an intake; a fan structure coupled with the combination supply/intake diffuser including a motor mechanically coupled with fan blades; a return air plenum for supplying air to the fan structure via the combination supply/intake diffuser; at least one attachment for supporting the fan structure such that no additional stress is applied to the ceiling; wherein the fan structure moves air through the intake to the supply.

Changes may be made in the above methods and systems without departing from the scope hereof. It should thus be noted that the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A system for air destratification and circulation, comprising:
an intake positioned proximate a ceiling of a space formed by a floor, a plurality of walls and the ceiling;
an exhaust, positioned proximate the ceiling and separate from the intake;
a fan structure comprising a motor mechanically coupled with fan blades;
a first duct for coupling the intake to the fan structure;
a second duct for coupling the exhaust to the fan structure; and
at least one attachment for supporting the fan structure such that no additional stress is applied to the ceiling from the fan structure;
wherein the fan structure moves air from the intake to the exhaust via the first and second ducts.

2. The system of claim 1, wherein the intake comprises an intake chosen from the group of intakes consisting of: perforated grille, perforated filter grille, T-Bar return air grille, return diffuser, return grille, return air grille, return air filter grille, bar-style grille, bar-style filter grille, egg crate return grille, and egg crate filter grille; wherein the intake is configured to replace at least a part of the ceiling.

3. The system of claim 1, wherein the exhaust comprises a diffuser configured to provide a column of air into the space.

4. The system of claim 1, wherein the exhaust is an exhaust chosen from the group of exhausts consisting of: jet diffuser, ball diffuser, supply diffuser, grille, linear bar diffuser, linear slot diffuser, fixed pattern diffuser, curved blade diffuser, nozzle diffuser, and supply register.

5. The system of claim 1, wherein one or more of the fan structure, the first duct and the second duct comprises acoustic properties for reducing noise produced by the system.

6. The system of claim 1 further comprising an ultraviolet germicidal irradiation (UVGI) device to purify air flowing through the system.

7. The system of claim 6 further comprising an air filter to purify air flowing through the system.

8. The system of claim 1, wherein the motor is an externally wound Permanent Split Capacitor (PSC) motor.

9. The system of claim 1, wherein the motor is one of an Electrically Commutated (EC) motor or a Direct Current (DC) motor.

10. The system of claim 1, wherein the fan blades are one or more of axial blades, mixed-flow blades, or centrifugal blades.

11. The system of claim 1, wherein one or both of the first and second ducts are insulated, non-insulated, rigid or flexible.

12. The system of claim 1, further comprising at least one additional exhaust coupled with the fan structure.

13. The system of claim 12, further comprising a valve configured to control airflow through the additional exhaust.

14. An air destratification, circulation, and/or purification control system, comprising:
a first air characteristic sensor for sensing an air characteristic reading at a first location within a space formed by a ceiling, a floor and a plurality of walls;
a second air characteristic sensor for sensing the air characteristic reading at a second location within the space;
a user interface unit for generating a speed control signal based upon the air characteristic readings sensed by the first and second air characteristic sensors; and
a speed control unit having a transformer based power controller for controlling, based upon the speed control signal, speed of a destratification fan configured with an externally wound permanent split capacitor motor.

15. The system of claim 14, wherein the first location is proximate the ceiling of the space and the second location is proximate the floor of the space.

16. The system of claim 14, wherein the interface generates the speed control signal based upon a spatial distance between the first and second locations.

17. The system of claim 14, further comprising at least one additional destratification fan coupled to the speed control unit.

18. The system of claim 14, further comprising:
a third air characteristic sensor for sensing the air characteristic reading at a third location within the space;
a fourth air characteristic sensor for sensing the air characteristic reading at a fourth location within the space; and
at least one additional speed control unit having a transformer based power controller for controlling speed of a second destratification fan configured with an externally wound permanent split capacitor motor;
wherein the user interface unit generates a second speed control signal based upon the air characteristic readings sensed by the third and fourth air characteristic sensors; and
wherein the at least one additional speed control unit controls the speed of the second destratification fan based upon the second speed control signal.

19. The system of claim 14 further comprising at least one additional destratification fan configured with an electrically commutated (EC) or DC motor;
wherein the speed of at least one of the additional destratification fans configured with an EC or a DC motor is controlled directly based upon at least one additional speed control signal.

20. A system for air destratification and circulation, comprising:
a combination supply/intake diffuser positioned in a ceiling of a space having a supply surrounded by an intake;
a fan structure coupled with the combination supply/intake diffuser including a motor mechanically coupled with fan blades;
a return air plenum for supplying air to the fan structure via the combination supply/intake diffuser;
at least one attachment for supporting the fan structure such that no additional stress is applied to the ceiling;
wherein the fan structure moves air through the intake to the supply.
